(19) United States
(12) Patent Application Publication
(10) Pub. No.: US 2022/0118164 A1
(43) Pub. Date: Apr. 21, 2022

(54) DEVICE FOR JOINT EXTRACTION OF A METAL CATION AND A TARGET MOLECULE

(71) Applicants: MEXBRAIN, Fontaines Saint-Martin (FR); UNIVERSITE CLAUDE BERNARD LYON 1, Villerubanne (FR); CENTRE NATIONAL DE LA RECHERCHE SCIENTIFIQUE - CNRS, Paris (FR)

(72) Inventors: Olivier TILLEMENT, Fontaines Saint-Martin (FR); François LUX, Lyon (FR); Marie VICTOR, Lyon (FR); Denise BECHET, Lyon (FR); Marco NATUZZI, Villeurbanne (FR); Jules Tillement, Fontaines Saint-Martin (FR); Thomas BRICHART, Lyon (FR)

(21) Appl. No.: 17/425,147

(22) PCT Filed: Jan. 24, 2020
(86) PCT No.: PCT/FR2020/050104

§ 371 Date: Jul. 22, 2021

(30) Foreign Application Priority Data
Jan. 25, 2019 (FR) 19 00699

(51) Int. Cl. A61M 1/36; A61K 47/69

(52) U.S. Cl. CPC A61M 1/362; A61M 1/1678; A61K 47/6935; A61K 47/6929

(57) ABSTRACT

The invention concerns the field of medical devices, more particularly devices for joint extraction, within an organism, of at least one metal cation and at least one target molecule. In order to do this, the device comprises: a) at least one ligand exhibiting specific affinity for the target molecule; b) at least one means for extraction of the metal cation, said means being a perfusion fluid comprising at least one chelating agent, the perfusion fluid being contained in a dialysis or microdialysis system. The use of these devices makes it possible, for example, to prevent and/or treat pathologies linked to dysregulation of the homeostasis of metals and/or target molecules in the organism, for example neurological diseases and/or proteinopathies.

### TECHNICAL FIELD

The invention relates to the field of medical devices, more particularly to devices enabling the joint extraction, from an organism, of at least one metal cation and at least one target molecule. The use of these devices makes it possible, for example, to prevent and/or treat pathologies linked to deregulation of the homeostasis of metals and/or of target molecules in the body, for example neurological diseases and/or proteinopathies.

### PRIOR ART

Maintaining the homeostasis of an organism's internal environment, meaning of all the biological fluids or liquids of the organism, is necessary to the proper functioning of the organism. Systemic or local dysregulation of the homeostasis of metals and/or peptides or proteins has been demonstrated in many diseases.

For metals, chelation therapies aimed at reducing the concentration of metal ions have already been in use for many years in cases of acute poisoning. A number of chelating agents are thus already accepted for use in humans, each associated with a particular group of metals (G. Crisponi et al., Coordination Chemistry Reviews, 2015).

More and more scientific studies highlight the important role that metals could have in a number of neurological impairments, in particular iron, but also copper, zinc, manganese, and even aluminum and lead (E. J. McAllum et al., J. Mol. Neurosci., 2016). This is particularly the case for neurodegeneration with iron overload which is a rare disease associated with a genetic anomaly linked to an accumulation of iron in certain areas of the brain and which so far has only been treated palliatively (S. Wiethoff et al., Handb. Clin. Neurol., 2017). In addition, many studies have shown that iron tends to accumulate in the brain with age (J. Acosta-Cabronero et al., Journal of Neuroscience, 2016). Wilson's disease is also a genetic disease, causing an accumulation of copper in the body and leading to various problems, particularly hepatic and/or neurological (Anna Cztonkowskal et al., Nature Rev., 2018).

Several neurological diseases such as Alzheimer's, Parkinson's, and Huntington's disease are also accompanied by an increase in the amount of iron in specific areas, leading to cellular damage as well as oxidative stress (A. A. Belaidi et al., Journal of Neurochemistry, 2016). For example, Huntington's disease is a neurodegenerative disease resulting in movement disorders, cognitive decline, and psychiatric problems. In this disease, many markers of oxidative stress are observed in the brain, which may be linked to a deregulation of iron homeostasis (S. J. A. van den Bogaard et al., International Review of Neurobiology, 2013). The increase in the level of iron in several regions of the brain (putamen, caudate nucleus, and pallidum) has thus been validated by several MRI studies including that of Bartzorkis (G. Bartzorkis et al., Archives of Neurology, 1999).

In these same pathologies, the homeostasis of other biological compounds is also disrupted. In Alzheimer's disease for example, the A-β (amyloid-beta) peptide, a peptide of about 42 amino acids (39 to 43), accumulates to form amyloid-beta aggregates. Treatments for amyloid diseases by extracting the Aβ peptide from biological fluids have thus been proposed (US2013/0045216 A1; M. Menendez-Gonzalez et al., Hypothesis and Theory, 2018). Still with the aim of treating or slowing the development of Alzheimer's disease, it has also been proposed to dilute the cerebrospinal fluid by replacing and filtering this fluid, in order to decrease the levels of Aβ peptide and of abnormally phosphorylated Tau protein (phospho-Tau) (M. M. Gonzalez, Cureus, 2017).

In addition to Alzheimer's disease, in many other amyloid pathologies such as Parkinson's disease and prion disease, a conformational conversion of normal soluble proteins into insoluble proteins has been demonstrated, leading to the formation of amyloid fibrils or plaques. Antibodies or small molecules specifically targeting these proteins are thus being studied with the aim being to inhibit the key stages of the aggregation process of these abnormal proteins, reduce the conversion of proteins into their pathological conformation, reduce the toxicity of pathological proteins, or increase the selective clearance of abnormal proteins (N. Cremades et al, Neurobiol Dis., 2018).

In addition, in Alzheimer's disease in particular, interactions have been demonstrated between Aβ peptides and certain metal ions, in particular ions from metals such as zinc, iron, or copper, which can lead to increased protein aggregation (Tougu et al. Metallomics, 2010).

It is thus accepted that in many proteinopathies, metal cations play an important role in the formation of abnormal configurations of certain proteins: in particular, some promote the formation of aggregates, fibrils, or other solid deposits. In proteinopathies, there would therefore locally be a two-fold deregulation of homeostasis: deregulation of the homeostasis of certain metals and deregulation of the homeostasis of protein-type target molecules, causing aggregates and other solid deposits.

Although scientific knowledge relating to these various pathologies is advancing (Pfaender S. et al., 2014; Boland B. et al., 2018; Iadanza M. G. et al., 2018), to date there is no effective treatment for Alzheimer's disease or Parkinson's disease, and more generally for neurodegenerative diseases and more inclusively for diseases involving multiple deregulations causing dyshomeostasis.

### Technical Problem

There is therefore currently a need to develop new means enabling the prevention and/or treatment of pathologies involving multiple deregulations causing dyshomeostasis and the formation of aberrant protein conformations leading to the formation of deposits, aggregates, fibrils, or plaques comprising said proteins. These means would thus have one or more of the following advantages:
a targeted and combined extraction of these compounds (target proteins and metals) within the body, whether they are present in high or low quantities,
an absence of toxicity,
when these compounds are co-aggregated in the body, an absence of releasing one or another of the compounds in isolation possibly occurring during the extraction process,
a determined duration of local efficacy not linked to the biodistribution of an administered drug,
a local action accessible even beyond the blood-brain barrier, in the case of treatment of neurological diseases,
an application suitable for the prevention and/or treatment of any pathology linked to deregulation of the homeostasis of target compounds, in particular associating metals and target molecules capable of co-aggregating.

These advantages and many others are described in the present disclosure.

### DISCLOSURE OF THE INVENTION

A device is proposed for the joint extraction of at least one metal cation and at least one target molecule from a biological fluid, a biological aggregate, an organ, or tissue, for diagnostic or therapeutic purposes, characterized in that it comprises:
a. at least one ligand exhibiting specific affinity for the target molecule;
b. at least one means for extraction of the metal cation, said means being a perfusion fluid comprising at least one chelating agent, said perfusion fluid being contained in a dialysis or microdialysis system.

Also proposed is a microdialysis system comprising said device for extraction and such that said system comprises at least:
a perfusion reservoir 7 comprising the perfusion fluid 11 and a collection reservoir 8 comprising the perfusion fluid comprising the extracted compounds 12; or a combined reservoir 22 comprising the perfusion fluid 11, 12;
a two-way catheter 3 connecting the microdialysis probe 1 to the perfusion reservoir 7 and collection reservoir 8 or to the combined reservoir 22;
a microdialysis probe 1 comprising a first lumen 4 allowing the passage of perfusion fluid 11 to a second lumen 5, said second lumen 5 allowing the discharge of perfusion fluid comprising the extracted compounds 12, and a microdialysis membrane 2 between the second lumen 5 and the exterior of the microdialysis probe 1 in contact with the biological fluid.

According to an alternative embodiment, a dialysis system is provided comprising said device for extraction and such that it comprises at least:
a probe with separate lumens 17, comprising a second catheter 5a allowing biological fluid to enter the dialysis system and a first catheter 4a allowing biological fluid to exit the dialysis system;
a reservoir 23 comprising: i) the perfusion fluid 11; ii) a dialysis compartment comprising a dialysis membrane 18 separating the perfusion fluid 11 from the biological fluid, the biological fluid entering said dialysis compartment via the second catheter 5a and exiting via the first catheter 4a.

The features set forth in the following paragraphs may optionally be implemented. They may be implemented independently of one another or in combination with one another.

The means for extraction of the metal cation may be a perfusion fluid used in a dialysis or microdialysis system, said perfusion fluid further comprising said ligand exhibiting specific affinity for the target molecule.

Advantageously, the complexation constant log(KC1) of the chelating agent for at least one metal cation is greater than 10, preferably greater than or equal to 15, and said at least one cation is selected from the cations of metals Cu, Fe, Zn, Hg, Cd, Pb, Mn, Co, Gd, and Al, alone or in combination, and more particularly Cu, Fe, and Zn, alone or in combination.

According to a preferred embodiment, the means for extraction makes it possible to extract the cations from a biological fluid, biological aggregate, organ, or tissue, when the content of said metal cations is less than 1 ppm, preferably less than 0.1 ppm, more preferably less than 0.01 ppm, and even more preferably less than 1 ppb.

The means for extraction may make it possible to extract a quantity of metal cations representing at least 1% of its mass, and preferably more than 10% of its mass.

The device is advantageously a perfusion fluid comprised in a dialysis system comprising a dialysis membrane and a reservoir comprising the perfusion fluid, said perfusion fluid being selected among:
a solution of nanoparticles comprising as active ingredient at least one chelating agent, and a solution of at least one ligand exhibiting specific affinity for the target molecule, the average diameter of said nanoparticles and of the ligand being greater than the pores of the dialysis or microdialysis membrane,
a solution of polymers, said polymers being grafted with at least one active ingredient which is a chelating agent, and a solution of at least one ligand exhibiting specific affinity for the target molecule, the average diameter being greater than the pores of said dialysis or microdialysis membrane.

Preferably, the device comprises a dialysis system comprising a dialysis membrane and a reservoir comprising a perfusion fluid, said perfusion fluid being selected among:
a solution of nanoparticles comprising as active ingredient at least one chelating agent and a solution of at least one ligand exhibiting specific affinity for the target molecule, the average diameter of said nanoparticles and of the ligand being greater than the pores of the dialysis membrane,
a solution of polymers, said polymers being grafted with at least one active ingredient which is a chelating agent, and a solution of at least one ligand exhibiting specific affinity for the target molecule, the average diameter being greater than the pores of said dialysis membrane.

According to an advantageous embodiment, the chelating agents are obtained by grafting, onto the nanoparticles or onto the polymer, one of the following complexing molecules or derivatives thereof: DOTA, DTPA, EDTA, EGTA, BAPTA, NOTA, DOTAGA, DFO, DOTAM, NOTAM, DOTP, NOTP, TETA, TETAM, TETP and DTPABA, or mixtures thereof.

Preferably, the nanoparticles are nanoparticles based on polysiloxane with an average diameter of between 3 and 50 nm, comprising the chelating agent obtained by grafting DOTA, DOTAGA, EDTA, or DTPA onto the nanoparticles.

According to one particular embodiment, the chelating agent contains at least one alkaline earth cation, preferably a cation of metals selected among Ca and Mg.

Advantageously, at least 10% of the chelating agents of said device are precomplexed with an alkaline earth cation; preferably 20%, more preferably 30%, and even more preferably more than 50% of the chelating agents of said device are precomplexed with an alkaline earth cation.

According to one embodiment, the nanoparticles or polymers, comprising the chelating agent obtained by grafting DOTA, DOTAGA, EDTA, or DTPA, have an average diameter greater than 20 kDa and less than 1 MDa.

Advantageously, the nanoparticles are based on polysiloxane or the polymers are based on chitosan or polyethylene glycol or polyvinyl alcohols.

According to one embodiment, the target molecule is selected among proteins, peptides, and glycoproteins. Advantageously, the target molecule is selected among amyloidogenic proteins and components of amyloid structures (in native monomeric form, or in the form of oligomers, or fibrils or aggregates or molecules responsible for their formation or their accumulation).

According to a preferred embodiment, the target molecule is selected among molecules involved in amyloidoses, tauopathies, or any pathology involving a deposit based on one or more proteins. Advantageously, the target molecule is selected among proteins and/or their precursors such as immunoglobulin light and heavy chains, serum amyloid A protein, transthyretin, apolipoprotein AI, AII, AVI, CII, or CIII, beta-2 microglobulin, gelsolin, lysozyme, fibrinogen, cystatin C, atrial natriuretic factor, calcitonin, amylin, insulin, prolactin, lactoferrin, cadherin, ABri, ADan, amyloid-beta peptide, prion protein, alpha-synuclein, tau protein, superoxide dismutase, huntingtin, neuroserpin, actin, ferritin, or mixtures thereof.

Preferably, the ligand is an antibody or an engineered protein ligand of the target molecule. Advantageously, the ligand is selected among:
antibodies targeting the amyloid-beta protein, preferably Solanezumab, Aducanumab, Crenezumab, Ponezumab, GSK933776, Gantenerumab, AAB-003, AAB-001, BAN2401, LY2599666, LY3002813, LY33887299322, SARI1014;
antibodies targeting alpha-synuclein, preferably BII054 or PRX002;
antibodies targeting the tau protein, preferably BII076, BII092, ABBV-8E12, JNJ-63733657, LY3303560, RG7345, R07105705, UCB0107;
antibodies targeting the serum amyloid A protein, preferably Dezamizumab or GSK2398852, Miridesap or GSK2315698, GSK3039294;
antibodies targeting transthyretin, preferably PRX004;
aptamers;
engineered protein ligands exhibiting specific affinity for at least one of the molecules, optionally selected among: ABD, Adhiron, Adnectin, Affibody, Affilin, Affimer, Affitin, Alphabody, Anticalin, Armadillo repeat proteins, Atrimer/tetranectin, Avimer/Maxibody, Centyrin, DARPin1;
engineered protein ligands of less than 50 kDA, preferably less than 30 kDa, and more preferably less than 5 kDa, grafted onto a nanoparticle or a polymer of more than 5 nm in hydrodynamic diameter, preferably more than 100 kDa, or
mixtures thereof.

According to a preferred embodiment, the device is used in the treatment of:
systemic and/or localized amyloidoses; in particular amyloidoses selected among: type AL amyloidosis (immunoglobulin light chain), type AH amyloidosis (immunoglobulin heavy chain), AA amyloidosis (serum amyloid A protein), ATTR amyloidosis (transthyretin), amyloid heart disease, renal amyloidosis, type II diabetes, prion diseases, or diseases linked to an amyloid protein,
tauopathies, in particular tauopathies selected among: Alzheimer's disease, progressive supranuclear palsy, frontotemporal dementia;
pathologies presenting a deposit based on at least one protein;
diseases presenting a metal dyshomeostasis particularly Wilson's disease or neurological disorders without amyloid characteristics such as autism or schizophrenia, . . . ), or neurological disorders with amyloid characteristics such as amyloidoses affecting or not affecting the central and/or peripheral nervous system.

### BRIEF DESCRIPTION OF DRAWINGS

Other features, details and advantages of the invention will be apparent from reading the detailed description below, and from analyzing the accompanying drawings, in which:

FIG. 1 shows a dialysis system according to one embodiment of the invention, said system comprising a dialysis probe 1, 17 placed in the brain. In such an embodiment, the biological fluid is cerebrospinal fluid.

FIG. 2 shows a dialysis system according to another embodiment of the invention, said system comprising a dialysis or microdialysis probe 1, 17 placed in the spine in cerebrospinal fluid. In such an embodiment, the biological fluid is advantageously spinal fluid.

FIG. 3 shows a microdialysis system according to one embodiment of the invention, said system comprising: i) a microdialysis probe 1 with a first lumen 4, a second lumen 5, and a microdialysis membrane 2 between the second lumen 4 and the exterior of the microdialysis probe 1 in contact with the biological fluid; ii) a perfusion reservoir 7 comprising the perfusion fluid 11 and a collection reservoir 8 comprising the perfusion fluid comprising the extracted compounds 12; iii) a two-way catheter 3 connecting the microdialysis probe 1 to the perfusion 7 and collection 8 reservoirs which are contained in a microdialysis housing 6.

FIG. 4 shows a system according to a second embodiment of the invention, said system comprising a microdialysis probe 1, a two-way catheter 3, and a combined reservoir 22. Said combined reservoir 22 has the function of containing a given volume of perfusion fluid whose composition in extracted compounds (metal cations and target molecules) increases with the cycles of fluid passing through the system.

FIG. 5 shows a dialysis system according to a third embodiment, said system comprising at least:
a probe with separate lumens 17, comprising a second catheter 5a allowing biological fluid to enter the system and a first catheter 4a allowing biological fluid to exit the system;
an extraction fluid reservoir 23 comprising
i) a perfusion fluid 11;
ii) a dialysis compartment comprising a dialysis membrane 18 separating the perfusion fluid 11 from the biological fluid, the biological fluid entering said dialysis compartment via the second catheter 5a and exiting via the first catheter 4a.

### DETAILED DESCRIPTION

For the most part, the drawings and the description below contain elements that are certain in nature. They can therefore serve not only to better understand the invention, but also contribute to its definition where appropriate.

The inventors have developed a medical device enabling the joint extraction of at least one metal cation and at least one target molecule, preferably at least two target molecules, from a fluid, a biological aggregate, an organ, or tissue, for diagnostic or therapeutic purposes.

The term “joint extraction of at least one metal cation and at least one target molecule” is understood to mean the simultaneous extraction of said metal cation and of said target molecule or the successive extraction of said metal cation and of said molecule target in any order whatsoever, the extraction of these two compounds being carried out with a short period of time between the two extractions, meaning preferably less than 24 hours, more preferably less than 12 hours, even more preferably less 1 hour.

The term “a metal cation” is understood to mean at least one metal cation. If several metal cations are involved, they may be metal cations of the same type or of different types.

The term “a target molecule” is understood to mean at least one target molecule. If several target molecules are involved, they may be target molecules of the same type or of different types.

The term “ligand” is understood to mean a molecule which binds, preferably reversibly, to the target molecule in a specific manner. Advantageously, the specific binding of ligand-target molecule is achieved by virtue of forces between molecules, such as ionic bonds, hydrogen bonds, hydrophobic interactions, and van der Waals forces. The ligand-target molecule interaction is thus reversible and more or less strong depending on the number and nature of the bonds formed. The strength of this interaction is defined by the affinity for the target molecule, which is linked to the dissociation constant.

The extraction of said biological compounds, meaning of said metal cation and of said target molecule, has the goal of maintaining homeostasis in said compounds, for therapeutic or diagnostic purposes. Maintaining homeostasis means regulating the content of said compounds within an organism, in particular with the aim being to extract said compounds in excess, which can be responsible for pathologies. Said compounds may be in excess within a biological fluid or within a biological aggregate. The extraction of one of the components may also have the aim of bringing the concentration of at least one of the biological compounds to below the solubility threshold of the biological aggregates linked to the pathology and thus to reduce their formation and/or lead to their dissolution.

The term “biological fluid” is understood to mean any fluid produced by the organism to which it relates. It may or may not be a circulating fluid. More particularly, it may be blood, lymph, bone marrow, chyle, any interstitial fluid, cerebrospinal fluid (CSF) or more specifically cerebrospinal fluid or spinal fluid, synovial fluid, peritoneal fluid.

The term “biological aggregate” is understood to mean any accumulation of target molecules and/or metals in the form of fibrils, matrix compounds, or plaques. As an example, they may be amyloid components, for example in the form of fibrils or plaques, accumulations of tau proteins, fatty plaques in particular such as atheromatous plaques, etc.

According to the invention, the term “organ” means all organs which can be brought into contact with the device of the invention or within which said device can be implanted or inserted. Preferably, the organ(s) are selected among the brain, the liver, the pancreas, the intestines, and the lungs.

According to the invention, the term “tissue” means all tissues which can be brought into contact with the device of the invention or within which said device can be implanted or inserted. Preferably, the tissue or tissues are selected among the peritoneum and tumor tissue (where appropriate from a tumor). For example, said device can be placed in contact, inserted, or implanted by endoscopy, in particular within a tumor.

“At least one” is understood to mean one or more of the compounds in question, of the same type or of a different type.

The term “dialysis” is also understood to mean specific dialyses such as, for example, microdialysis.

The extraction device comprises a ligand exhibiting specific affinity for the target molecule. Thus, said compound is able to bind specifically to said target molecule. It may be an antibody, a nanobody, a peptide, a protein, or any other ligand able to bind specifically to the target molecule.

The term “antibody” is understood to mean an immunoglobulin formed of 4 polypeptide chains, two heavy H and two light L, capable of specifically binding an antigen, also called a target molecule in the context of the invention.

The term “nanobody” is understood to mean an antibody element capable of specifically binding an antigen or target molecule in the context of the invention.

“Engineered protein ligand” (“scaffold protein” or “engineered protein”) is understood to mean a compound or protein fragments selected for their affinity towards specific target molecules. They are generally lighter than antibodies, often easier to produce as well, and chemically stable. Advantageously, the engineered protein ligands are less than 50 kDA, preferably less than 30 kDa, and more preferably less than 3 kDa. Such ligands have a good specific surface area. These engineered protein ligands may be selected among: ABD, Adhiron, Adnectin, Affibody, Affilin, Affimer, Affitin, Alphabody, Anticalin, Armadillo repeat proteins, Atrimer/tetranectin, Avimer/Maxibody, Centyrin, DARPin1.

The extraction device further comprises a means for extraction of at least one metal cation, said means being a perfusion fluid comprising at least one chelating agent, said perfusion fluid being contained in a dialysis system.

According to the invention, the term “chelating agent” means an organic group capable of complexing with at least one metal cation. The complexation reaction can be a transmetalation, meaning an exchange of two metal cations. In such a case, the chelating agent may be precomplexed with a first metal cation which will subsequently be exchanged with the target metal cation.

In an advantageous embodiment, at least 10% of the chelating agents of said device are precomplexed with an alkaline earth cation, preferably 20%, more preferably 30%, and even more preferably more than 50% of the chelating agents of said device are precomplexed with an alkaline earth cation.

According to a preferred embodiment, the complexation constant log(KC1) of said chelating agent for at least one of said metal cations is greater than 10, in particular 11, 12, 13, 14, 15, and is preferably greater than or equal to 15. When the chelating agent is precomplexed with a first metal cation, the complexation constant log(KCl′) for the first metal cation is less than the complexation constant log(KCl) of the target metal cation.

Advantageously, the chelating agent, with a constant at least greater than or equal to 10 and preferably greater than or equal to 15, complexes at least one of the cations of the metals Copper (Cu), Iron (Fe), Zinc (Zn), Mercury (Fig), Cadmium (Cd), Lead (Pb), Aluminum (Al), Manganese (Mn), Arsenic (As), Mercury (Hg), Cobalt (Co), Nickel (Ni), Vanadium (V), Tungsten (W), Zirconium (Zr), Titanium (Ti), Chromium (Cr), Silver (Ag), Bismuth (Bi), Tin (Sn), Scandium (Sc), Yttrium (Y), Lanthanum (La), Cerium (Ce), Praseodymium (Pr), Neodymium (Nd), Samarium (Sm), Europium (Eu), Gadolinium (Gd), Terbium (Tb), Dysprosium (Dy), Holmium (Ho), Erbium (Er), Thulium (Tm), Ytterbium (Yb), Lutecium (Lu), Actinium (Ac), Uranium (U), Plutonium (Pu), Americium (Am), alone or in combination. Even more advantageously, the chelating agent complexes at least one of the cations of the metals Copper, Iron, Zinc, Mercury, Cadmium, Lead, Aluminum, Manganese, and Gadolinium, in particular Manganese and Gadolinium. Even more advantageously, the chelating agent complexes at least one of the cations of the metals Copper, Iron, and Zinc, alone or in combination.

Advantageously, the specificity of the chelating agent for said metal cation to be extracted is high relative to the other cationic trace elements, in particular the difference between the complexation constants is preferably greater than 3; and, more particularly, the difference between the complexation constants with calcium and magnesium is preferably greater than 3 and even greater than 5.

According to a preferred embodiment, said device, or more specifically the means for extraction of at least one metal cation, also contains trace elements selected among calcium, magnesium, iron, copper, zinc, and manganese within the perfusion fluid or even directly on said chelating agent. In the latter case, this involves a transmetalation reaction and the cations are specifically chosen to allow such a reaction. Such an embodiment makes it possible, for example, to regulate the homeostasis of essential metals. The chelating agent contains at least one alkaline earth cation, preferably a cation of metals selected among Ca and Mg.

According to one embodiment of the invention, the means for extraction of the metal cation allows extracting said metal cation from a biological fluid, a biological aggregate, an organ, or tissue, when its content is less than 1 ppm, in particular 0.1 ppm, 0.01 ppm, and is preferably less than 1 ppb.

According to an advantageous embodiment, said means for extraction of the metal cation allows extracting a quantity of metal cations representing at least 1% of its mass, and preferably more than 10% of its mass.

In addition, the extraction device comprises at least one ligand exhibiting specific affinity for a target molecule. Said target molecule is preferably selected among proteins, peptides, and glycoproteins, more preferably it is selected among components of amyloid structures. According to an advantageous embodiment, the target molecule comprises a specific peptide sequence recognized by the ligand.

According to one embodiment of the invention, the target molecule is selected among proteins, peptides, and glycoproteins.

Advantageously, the target molecule is selected among amyloidogenic proteins and components of amyloid structures, in particular in native monomeric form or in the form of oligomers, fibrils, or biological aggregates. The target molecule may also be one or more molecules responsible for the formation or accumulation of said oligomers, fibrils, or biological aggregates.

According to a preferred embodiment, the target molecule is selected among molecules involved in amyloidoses, tauopathies, or any pathology presenting a deposit based on one or more proteins.

According to one embodiment compatible with the preceding embodiments, the target molecule is selected among proteins and/or their precursors such as immunoglobulin light and heavy chains, serum amyloid A protein, transthyretin, apolipoprotein AI, AII, AVI, CII, or CIII, beta-2 microglobulin, gelsolin, lysozyme, fibrinogen, cystatin C, atrial natriuretic factor, calcitonin, amylin, insulin, prolactin, lactoferrin, cadherin, ABri, ADan, amyloid-beta peptide, prion protein, alpha-synuclein, tau protein, superoxide dismutase, huntingtin, neuroserpin, actin, ferritin, or mixtures thereof.

Preferably, the ligand is an antibody or an engineered protein ligand of the target molecule. Advantageously, the ligand is selected among:
antibodies targeting amyloid-beta protein, preferably Aducanumab, Crenezumab, Ponezumab, GSK933776, Gantenerumab, AAB-003, AAB-001, BAN2401, LY2599666, LY3002813, LY3372993, MED11814, SAR228810,
antibodies targeting alpha-synuclein, preferably BII054 or PRX002;
antibodies targeting tau protein, preferably BII076, BII092, ABBV-8E12, JNJ-63733657, LY3303560, RG7345, R07105705, UCB0107;
antibodies targeting serum amyloid A protein, preferably: Dezamizumab or GSK2398852, Miridesap or GSK2315698, GSK3039294;
antibodies targeting transthyretin, preferably PRX004;
aptamers;
antibodies targeting molecules, in particular proteins, peptides and/or their precursors, involved in amyloidoses, tauopathies, or pathologies exhibiting a protein-based deposit; which is all proteins and their precursors;
engineered protein ligands, advantageously less than 50 kDA, preferably less than kDa, and more preferably less than 3 kDa, grafted onto a nanoparticle, preferably a polysiloxane nanoparticle, or a polymer more than 5 nm in hydrodynamic diameter, preferably more than 100 kDa, and advantageously less than 1 μm in hydrodynamic diameter or less than 1 MDa. Such ligands have a good specific surface area. In this embodiment, there may be one or more proteins per nanoparticle or per polymer. In addition, it is possible to envisage grafting one or more chelating agents onto the nanoparticle or polymer. These engineered protein ligands may be selected among: ABD, Adhiron, Adnectin, Affibody, Affilin, Affimer, Affitin, Alphabody, Anticalin, Armadillo repeat proteins, Atrimer/tetranectin, Avimer/Maxibody, Centyrin, DARPin1, or
mixtures thereof.

According to one embodiment, the extraction device comprises a perfusion fluid comprising at least one chelating agent, said perfusion fluid being contained in a dialysis or microdialysis system or any miniaturized dialysis device, in particular with a fixed exchange reservoir.

Advantageously and according to a preferred embodiment, the dialysis or microdialysis system comprises:
a. a semi-permeable dialysis 18 or microdialysis 2 membrane,
b. one or more reservoirs 7/8 or 22 or 23 comprising the perfusion fluid.

According to one embodiment, said means for extraction of the metal cation is a perfusion fluid used in a dialysis or microdialysis system which further comprises the ligand exhibiting specific affinity for the target molecule.

According to the invention, the term “dialysis system” means any system allowing the passage of metal cations and/or at least one target molecule of interest from the extraction device through a dialysis 18 or microdialysis 2 membrane semipermeable to water and to the cations and/or molecules mentioned above.

The term “microdialysis system” is understood to mean a very small-scale dialysis system. For example, a microdialysis technique requires the insertion of a small microdialysis catheter, also called a microdialysis probe 1, into the tissue. The microdialysis probe is designed to mimic a blood capillary and consists of a tube with a semi-permeable membrane at its end, such as a hollow fiber membrane, which is connected to the inlet and outlet tubing. Microdialysis makes it possible to extract or deliver only the compounds capable of passing through a semi-permeable membrane whose cut-off threshold is selected according to the intended application. In the case of dialysis, this is often a dynamic diffusion phenomenon, guided by the difference in concentration of the diffusing species between each side of the dialysis membrane.

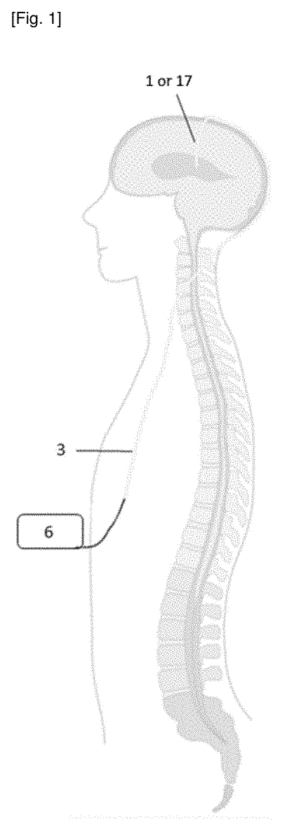

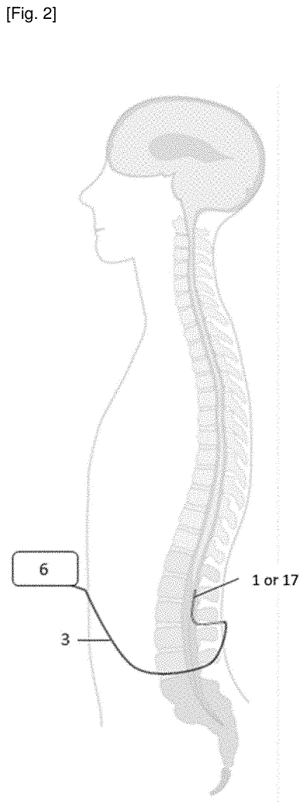

When using dialysis systems to extract compounds in low concentrations (metal cations and/or target molecules), the driving force is often quickly limited or saturated and the trapping of the compound(s) concerned is limited by the equilibrium concentration. Advantageously, a microdialysis system makes it possible to circumvent the problems of conventional chelating agents or ligands and to extract, locally or more generally, a very high proportion of the targeted metal cations (or of metal cations and target molecules), due to the maintaining inside the dialysis membrane of complexing chemical species (chelating agent(s) and/or ligand(s)) for at least one target metal cation and/or one target molecule. The chelating agents are advantageously grafted onto macromolecules or nanoparticles which have a mass greater than the cut-off threshold of the membrane, so that the complexing species remain within the perfusion fluid on one side of the dialysis membrane. Similarly, the ligands are advantageously present within or grafted onto macromolecules or nanoparticles which possess, or possess by their very nature, a mass greater than the cut-off threshold of the membrane. The dialysis system containing the complexing species is placed at the area of interest, for example at the brain (FIG. 1) in the case of treatment of neuro-degenerative diseases or near the spinal cord (FIG. 2). As the metal cations and/or target molecules are smaller than the cut-off threshold of the dialysis membrane, they will be able to diffuse through the membrane to the perfusion fluid comprising the chelating agents and/or ligands. The strong complexing properties of the chelating agents and/or ligands used will enable chelation of the target metals and/or target molecules even if they are present in very small amounts in the biological fluid. As an example, the target compounds (metal cations and/or molecules) may be present in small amounts in the biological fluid because they are in the form of biological aggregates. Chelation of the metal cations and/or binding with the target molecules will therefore decrease the concentration of the compounds to be extracted, in the solution inside the reservoir comprising the perfusion fluid, making it possible to maintain a strong gradient of concentration of compounds to be extracted across the dialysis 18 or microdialysis 2 membrane, thus making it possible to prolong the extraction and to maintain a flow of target compounds. In order not to disturb the homeostasis of other compounds such as other metal cations and other molecules present in the biological fluid, these elements may be comprised in the perfusion fluid in a concentration equivalent to that of the biological fluid.

Dialysis or microdialysis devices known to those skilled in the art may be used, on condition that they contain a semi-permeable dialysis membrane and a reservoir comprising a perfusion fluid containing at least one chelating agent and/or a ligand as mentioned above. As examples, devices which can be used are the medical devices developed by the companies M Dialysis AB, Sweden; Integra Life Sciences; in particular such as the microdialysis catheters (references 8010509, P000049, 8010337, this list not being exhaustive).

Dialysis systems, advantageously compact dialysis or microdialysis systems, can be used with an exchange reservoir of fixed volume 22 or 23. The specific capture of the cations to be extracted due to the chelating agent and of the target molecule due to the ligand makes it possible to maintain a purification gradient between the biological fluids to be purified and the perfusion fluid, even for small volumes of fluids with no circulation. In such an embodiment, the components of the biological fluids which one wishes to purify are preserved.

According to one embodiment, the dialysis system comprises a dialysis membrane 18 and a reservoir with a fixed volume 23, preferably with no circulation of fluid. Advantageously, the reservoir has a volume of less than 100 ml, preferably less than 20 ml, and more preferably less than 10 ml. Such a dialysis system is particularly suitable for a biological fluid such as cerebrospinal fluid.

Advantageously, and according to a preferred embodiment, the dialysis or microdialysis system comprises a microdialysis probe 1 continuously perfused with a perfusion fluid in the form of an aqueous solution (perfusate) which resembles the composition (ionic and/or molecular) of the surrounding biological fluid, at a low flow rate of less than 1 mL/min and preferably less than 0.1 mL/min. According to another embodiment, the means for extraction comprises a dialysis probe 1 continuously perfused with a perfusion fluid at a flow rate of less than 10 mL/min and preferably between 1 and 5 mL/min.

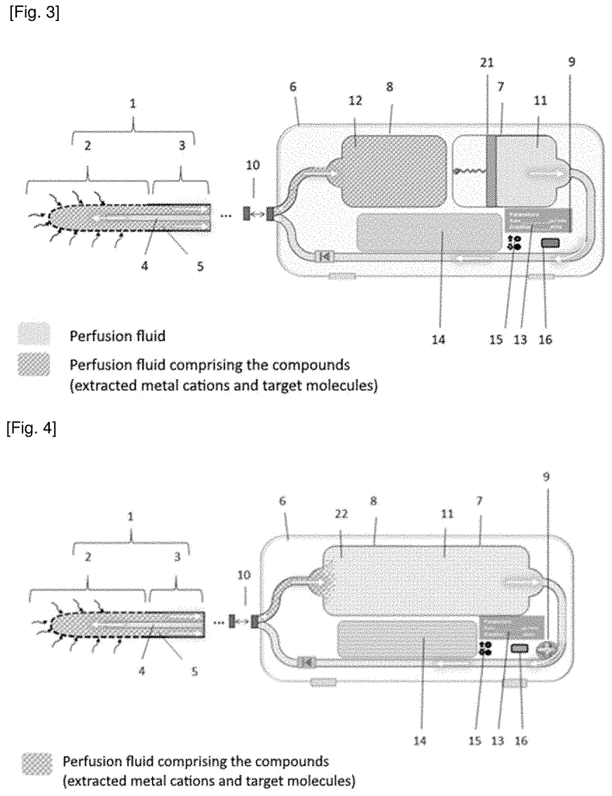

In one embodiment illustrated in FIG. 3, the system comprises at least:
a perfusion reservoir 7 comprising the perfusion fluid 11 and a collection reservoir 8 comprising the perfusion fluid comprising the extracted compounds 12 (metal cations and target molecules);
a two-way catheter 3 connecting the microdialysis probe 1 to the perfusion reservoir 7 and to the collection reservoir 8;
a microdialysis probe 1 comprising: i) a first lumen 4 allowing the passage of perfusion fluid 11 to a second lumen 5, said second lumen 5 allowing the discharge of perfusion fluid comprising the extracted compounds 1 to the collection reservoir 8, and ii) a microdialysis membrane 2 between the second lumen 5 and the exterior of the microdialysis probe 1 in contact with the biological fluid.

The microdialysis probe is preferably a linear or concentric probe. According to one embodiment illustrated in FIG. 3, the microdialysis probe is a concentric probe, the first lumen 4 advantageously being comprised inside the second lumen 5. Such a dialysis or microdialysis system not only allows the extraction of metal cations and target molecules but also the performance of quantitative and/or qualitative analyses of the biological fluid.

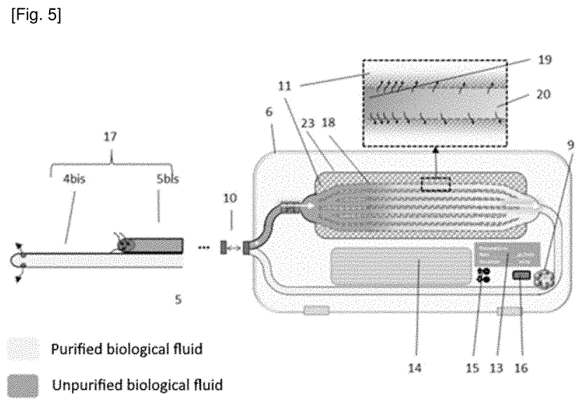

In another embodiment illustrated in FIG. 4, the system comprises a microdialysis probe 1 as well as a two-way catheter 3, as in the previous embodiment. The system further comprises a single reservoir called a combined reservoir 22, rather than a perfusion reservoir and a collection reservoir. The function of said combined reservoir 22 is to contain a given volume of perfusion fluid in which the concentration of extracted compounds (metal cations and target molecules, free and/or bound or/and chelated) increases with the cycles of fluid passing through the device. Advantageously, the dialysis or microdialysis system is provided with a system for replacing the perfusion fluid in the combination reservoir 22. After several cycles of fluid passing through, the compound-saturated fluid can then be replaced by a perfusion fluid without compounds to be extracted.

In another embodiment, illustrated in FIG. 5, the dialysis system comprises:
a probe with separate lumens 17, comprising a second catheter 5a allowing biological fluid to enter the system (labeled “unpurified biological fluid 19”—FIG. 5) and a first catheter 4a allowing biological fluid to exit the system (labeled “purified biological fluid 20”—FIG. 5);
a reservoir 23 comprising: i) a perfusion fluid 11; ii) a dialysis compartment comprising a dialysis membrane 18 separating the perfusion fluid 11 from the biological fluid 19, 20, the biological fluid entering said dialysis compartment via the second catheter 5a and exiting via the first catheter 4a. The two-way catheter 3 enables the connection between the probe with separate lumens 17 and the reservoir 23 contained in the compact dialysis housing 6. Advantageously, the perfusion fluid 11 is non-circulating. A device advantageously allows occasional replacement of said perfusion fluid 11, meaning at given time intervals, when the free concentration of metal cations and/or of target molecules is in equilibrium or close to equilibrium with that of the biological fluid. According to one embodiment, the dialysis compartment is placed within the perfusion fluid.

According to an advantageous embodiment compatible with any one of the preceding embodiments, the reservoirs 7/8, 22 or 23 are comprised in a housing 6 further comprising at least one of the following elements:
a miniaturized pump 9;
a lockable connection system 10 capable of allowing circulation or stopping circulation of fluid in the catheter(s) 3; optionally said lockable connection system is capable of allowing a disconnection of the housing 6 and of the probe 1 or 17 for example in order to be able to replace one of the two independently;
at least one sensor 16 capable of enabling measurement of the speed of the fluid within the dialysis or microdialysis system, the pressure inside said system, or any other parameter relating to the fluid, for example its temperature and/or its composition;
an electronic control/recording system 15 capable of calibrating the parameters of flow rate or perfusion duration, for example to allow an action on the pump or pumps 9, comprising for example a circuit board 14 and a control screen 13 capable of displaying the parameters measured within the dialysis or microdialysis system,
a syringe pump 21, preferably comprised in the perfusion reservoir 7, in order to circulate the perfusion fluid 11 inside the system;
possibly a device for regulation and communication with the outside world.

According to a preferred embodiment, the perfusion fluid comprises: i) a solution of nanoparticles comprising as active ingredient at least one chelating agent, and ii) a solution of at least one ligand exhibiting specific affinity for the target molecule, the average diameter of said nanoparticles and of the ligand being greater than the pores of the dialysis 18 or microdialysis 2 membrane. In one aspect, the cut-off threshold of the porous dialysis 18 or microdialysis 2 membrane is less than the mass of the chelating agent, i.e. the mass of the nanoparticle comprising at least one chelating agent.

Alternatively, the perfusion fluid comprises a solution of polymers, said polymers being grafted with at least one active ingredient which is a chelating agent, and a solution of at least one ligand exhibiting specific affinity for the target molecule, the average diameter being greater than the pores of said dialysis or microdialysis membrane. In this aspect, the cut-off threshold of the dialysis 18 or microdialysis 2 membrane is less than the mass of the chelating agent, i.e. the mass of the polymer onto which at least one chelating agent is grafted.

According to the invention, the term “solution” means a mixture of liquid and solid particles which remain evenly dispersed, the particles often being sufficiently small (microscopic or nanoscopic) for the mixture to remain stable and homogeneous.

According to one advantageous embodiment, the perfusion fluid is an “artificial cerebrospinal fluid” type of liquid comprising chelating agents based on polysiloxane and/or chitosan. Advantageously, it contains about 1 to 10 millimoles per liter of chelating agents of type EDTA, DTPA, and DOTA. According to a preferred embodiment, it may be one of the MetAEx® or proMetAEx® solutions marketed by Mexbrain.

According to one embodiment, said average diameter is greater than the pores of said dialysis or microdialysis membrane by at least 10%, 20%, 30%, 40%, 50%, 60%, 70%, 80%, 90%, or 100%.

According to the invention, the term “average diameter” means the harmonic mean of the diameters of the compounds, in particular of nanoparticles or of polymers, of ligands. The compound size distribution is for example measured using a commercial particle size analyzer, such as a Malvern Zetasizer Nano-S particle size analyzer based on PCS (Photon Correlation Spectroscopy) which is characterized by an average hydrodynamic diameter. A method of measuring this parameter is also described in the 1996 ISO 13321 standard.

In one embodiment, the solution contains more than 1% by mass of nanoparticles or polymers, in particular more than 2%, 3%, 4%, 5%, 6%, 7%, 8%, 9%, and preferably more than 10% by mass.

According to one embodiment, the chelating agent can be grafted onto said ligand specific for the target molecule. The chelating agents can thus be directly attached to the ligands, for example by covalence and covalent bonds, and more particularly by peptic bonds.

The nanoparticles which can be used in the extraction device advantageously comprise two characteristics:
they are based on polysiloxane or carbon,
they have an average hydrodynamic diameter greater than 3 nm, and preferably less than 50 nm.

In one embodiment, the nanoparticle comprises, as active ingredient, at least one chelating agent capable of complexing the metal cations, said chelating agent having a complexing constant log(KC1) for at least one of said metal cations that is greater than 10, and preferably greater than or equal to 15.

According to the invention, the term “silica-based nanoparticles” means nanoparticles characterized by a mass percentage of silicon of at least 8%.

According to the invention, the term “polysiloxane-based nanoparticles” means nanoparticles characterized by a mass percentage of silicon of at least 8%.

According to the invention, the term “polysiloxane” means an inorganic crosslinked polymer consisting of a chain of siloxanes.

The structural units of polysiloxane, which are identical or different, are of the following formula:

Si(OSi)nR4-n

where:
R is an organic molecule linked to silicon by a covalent Si—C bond
n is an integer between 1 and 4.

As a preferred example, the term “polysiloxane” includes in particular the polymers resulting from condensation of tetraethyl orthosilicate (TEOS) and of aminopropyltriethoxysilane (APTES) by a sol-gel process.

Advantageously, said nanoparticle thus comprises:
a. polysiloxanes, with a silicon mass ratio of at least 8% of the total mass of the nanoparticle, preferably between 8% and 50% of the total mass of the nanoparticle,
b. chelating agents, preferably in a proportion of between 5 and 1000, and preferably between 50 and 500, per nanoparticle,
c. where appropriate, metal elements, for example in a proportion of between 50 and 500, and preferably between 100 and 200, per nanoparticle, said metal elements being complexed with the chelating agents.

In one embodiment, the nanoparticles which can be used according to the invention do not comprise metal elements. In other words, in the above definition, said nanoparticle comprises only elements a. (polysiloxanes or silicon) and b. (chelating agents).

In one embodiment, the chelating agents complex the cations of metals Cu, Fe, Zn, Hg, Cd, Pb, Mn, Al, Ca, Mg, Gd.

In one embodiment, the chelating agents are obtained by grafting (covalent bond) onto the nanoparticle of one of the following complexing molecules or its derivatives, such as aminopolycarboxylic acids and their derivatives, in particular selected among: DOTA (1,4,7,10-tetraazacyclododecane-N,N′,N″,N′″-tetraacetic acid), DTPA (diethylenetriaminepentaacetic acid), D03A-pyridine of formula (I) below:

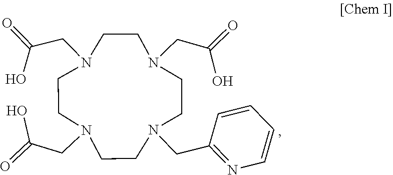 [SMILES: O=C(O)CN1CCN(CC(=O)O)CCN(Cc2ccccn2)CCN(CC(=O)O)CC1]

EDTA (2,2′,2″,2′″-(ethane-1,2-diyldinitrilo)tetraacetic acid), EGTA (ethylene glycol-bis(2-aminoethyl ether)-N,N,N′,N′-tetraacetic acid), BAPTA (1,2-bis(o-aminophenoxy)ethane-N,N,N′,N′-tetraacetic acid), NOTA (1,4,7-triazacyclononane-1,4,7-triacetic acid), DOTAGA ((2-(4,7,10-tris(carboxymethyl)-1,4,7,10-tetraazacyclododecan-1-yl)pentanedioic acid), DFO (deferoxamine), amide derivatives such as for example DOTAM (1,4,7,10-tetrakis(carbamoylmethyl)-1,4,7,10 tetraazacyclododecane) or NOTAM (1,4,7-tetrakis(carbamoylmethyl)-1,4,7-triazacyclononane), as well as mixed carboxylic acid/amide derivatives, phosphonic derivatives such as for example DOTP (1,4,7,10-tetraazacyclododecane-1,4,7,10-tetrakis(methylene phosphonate)) or NOTP (1,4,7-tetrakis(methylene phosphonate)-1,4,7-triazacyclononane), cyclam derivatives such as TETA (1,4,8,11-tetraazacyclotetradecane-N,N′,N″,N′″-tetraacetic acid), TETAM (1,4,8,11-tetraazacyclotetradecane-N,N′,N″,N′″-tetrakis(carbamoylmethyl)), TETP (1,4,8,11-tetraazacyclotetradecane-N,N′,N″,N′″-tetrakis(methylene phosphonate)), or mixtures thereof.

Preferably, said chelating agents above are linked directly or indirectly by covalent bond to the silicons of the polysiloxanes of the nanoparticle. The term “indirect” bond means the presence of a molecular “linker” or “spacer” between the nanoparticle and the chelating agent, said linker or spacer being covalently linked to one of the components of the nanoparticle.

According to a preferred embodiment, said nanoparticle is a polysiloxane-based nanoparticle with an average hydrodynamic diameter of between 3 and 100 nm, comprising the chelating agent obtained by grafting DOTA, DOTAGA, EDTA, or DTPA onto the nanoparticle.

According to a preferred embodiment, said nanoparticle is a nanoparticle with an average diameter greater than 20 kDa and less than 1 MDa, comprising the chelating agent obtained by grafting DOTA, DOTAGA, EDTA, or DTPA onto the nanoparticle.

According to a preferred embodiment, said solution comprising said nanoparticles also contains trace elements selected among Calcium, Magnesium, Iron, Copper, Zinc, or Manganese.

In another embodiment of the invention, polymers may be used in place of the aforementioned nanoparticles. In such case, said polymers are grafted with at least one chelating agent.

The term “polymer” is understood to mean any macromolecule formed from the covalent chaining of a very large number of repeating units which derive from one or more monomers. The polymers preferably used are, for example, from the family of chitosans, polyacrylamides, polyamines or polycarboxylics, polyethylene glycols, polyvinyl alcohols (PVA). For example, they can be polymers containing amino groups such as chitosan. According to a preferred embodiment, said polymer is biocompatible.

In one embodiment, the chelating agents or their derivatives grafted onto said polymers are aminopolycarboxylic acids and their derivatives, in particular selected among: DOTA, DTPA, DO3A-pyridine of formula (I) above, EDTA, EGTA, BAPTA, NOTA, DOTAGA, DFO, DOTAM, NOTAM, DOTP, NOTP, TETA, TETAM, and TETP or mixtures thereof.

Preferably, said chelating agents above are linked directly or indirectly by covalent bond to the polymer or to a polymer chain of more than 10 kDa and preferably more than 100 kDa. The term “indirect” bond means the presence of a molecular “linker” or “spacer” between the polymer and the chelating agent, said linker or spacer being covalently linked to one of the components of said polymer.

In one embodiment, the chelating agents or their derivatives grafted onto said polymers will comprise dithiocarbamate functional groups.

According to a preferred embodiment, said polymer grafted with a chelating agent is selected among: chitosan grafted with DPTA-BA or chitosan grafted with DFO or chitosan grafted with EDTA-BA or chitosan grafted with DOTAGA-A.

According to a preferred embodiment, said solution comprising said polymers also contains trace elements, selected among Calcium, Magnesium, Iron, Copper, Zinc, or Manganese.

Alternatively, the perfusion fluid is a solution of chelating molecules. Said chelating molecules may have an average diameter greater than the pores of said dialysis or microdialysis membrane, i.e. greater than the cutoff threshold of the membrane, in order to be retained within the liquid of the dialysis membrane. In another embodiment, they may have an average diameter smaller than the pores of said dialysis or microdialysis membrane, and in this case they can pass through the pores of the membrane before passing into the body and be naturally eliminated by the kidneys or liver.

### INDUSTRIAL APPLICATION

The invention may find applications in particular in maintaining homeostasis, particularly in maintaining the homeostasis of two target compounds such as a metal cation and a target molecule.

According to a preferred embodiment, the device for the joint extraction of at least one metal cation and at least one target molecule of a biological fluid or of a biological aggregate, mentioned above, is used in the treatment of a disease selected among:
systemic and/or localized amyloidoses; in particular amyloidoses selected among: type AL amyloidosis (immunoglobulin light chain), type AH amyloidosis (immunoglobulin heavy chain), AA amyloidosis (serum amyloid A protein), ATTR amyloidosis (transthyretin), amyloid heart disease, renal amyloidosis, type II diabetes, prion diseases, or diseases linked to an amyloid protein. Amyloidosis is also implicated in neurodegenerative diseases, in particular Alzheimer's disease (amyloid-beta), Parkinson's disease (alpha-synuclein), amyotrophic lateral sclerosis (superoxide dismutase), Huntington's disease (Huntingtin).
tauopathies, in particular: tauopathies selected among: Alzheimer's disease, progressive supranuclear palsy, frontotemporal dementia;
pathologies presenting a deposit based on at least one protein;
diseases presenting a metal dyshomeostasis particularly Wilson's disease or neurological disorders without amyloid characteristics such as autism or schizophrenia, or neurological disorders with amyloid characteristics such as the amyloidoses mentioned above affecting or not affecting the central and/or peripheral nervous system.

According to a preferred embodiment, the device for the joint extraction of at least one metal cation and at least one target molecule of a biological aggregate mentioned above is used to slow down the formation of, dissociate, or dissolve a biological aggregate, preferably in the form of oligomers, fibrils, or plaques comprising at least the target molecule; it is used in diagnosis, prevention, and/or therapy.

The invention also relates to a method for extracting metal cations and target molecules in a subject, comprising the administration of an implant onto which is grafted at least one chelating agent, or the use of a perfusion fluid containing at least one chelating agent within a device such as those mentioned above.

According to the invention, said “subject” is understood to mean a human or an animal in which prevention or treatment is to take place.

The invention is not limited to the preceding description, but encompasses all variants conceivable to a person skilled in the art within the framework of the protection sought.

### LIST OF REFERENCE SYMBOLS

1. Microdialysis probe
2. Microdialysis membrane
3. Two-way catheter
4. First lumen
4a. First catheter
5. Second lumen
5a. Second catheter
6. Compact housing
7. Perfusion reservoir
8. Collection reservoir
9. Pump
10. Lockable connection system
11. Perfusion fluid
12. Perfusion fluid comprising the extracted compounds (metal cations and target molecules)
13. Control screen (display of parameters)
14. Circuit board
15. Control of parameters
16. Sensors
17. Double-lumen probe
18. Dialysis membrane
19. Unpurified biological fluid
20. Purified biological fluid
21: Syringe pump
22: Combined reservoir
23: Reservoir

### LIST OF CITED DOCUMENTS

### Patent Documents

For all appropriate purposes, the following patent document(s) is (are) cited:
US2013/0045216 A1 (Appl. No.: U.S. Ser. No. 13/655,234)

### Non-Patent Literature

For all appropriate purposes, the following non-patent element(s) is (are) cited:
G. Crisponi, V. M. Nurchi, V. Bertolasi, M. Remelli, G. Faa, “Chelating agents for human diseases related to aluminium overload”, Coordination Chemistry Reviews, Volume 256, Issues 1-2, January 2012, pp. 89-104
E. J. McAllum and D. Finkelstein, “Metals in Alzheimer's and Parkinson's Disease: Relevance to Dementia with Lewy Bodies”, Journal of Molecular Neuroscience, Volume 60(3), August 2016, pp. 279-288]
S. Wiethoff and H. Houlden “Neurodegeneration with brain iron accumulation”, Handb. Clin. Neurol., Volume 145, 2017, pp. 57-166
J Acosta-Cabronero, Matthew J. Betts, Arturo Cardenas-Blanco, Shan Yang and Peter J. Nestor, “In Vivo MRI Mapping of Brain Iron Deposition across the Adult Lifespan”, Journal of Neuroscience, January 2016, 36 (2) pp. 364-374;
A. A. Belaidi and A. I. Bush, “Iron neurochemistry in Alzheimer's disease and Parkinson's disease: targets for therapeutics”, Journal of Neurochemistry, Vol. 139, Issue S1, 2016, pp. 179-197
Simon J. A. van den Bogaard, Eve M. Dumas, Raymund A. C. Roos “Metal Related Neurodegenerative Disease—International Review of Neurobiology, Volume 110, 2013, pp. 241-250,
https://www.sciencedirect.com/science/article/pii/B9780124105027000119,
G. Bartzokis, J. Cummings, S. Perlman, D. B. Hance, J. Mintz, Increased basal ganglia iron levels in Huntington disease, Arch. Neurol., 1999, 5, 569-574. https://jamanetwork.com/journals/jamaneurology/fullarticle/775016
M. Menéndez-Gonzalez, H. S. Padilla-Zambrano, G. Alvarez, E. Capetillo-Zarate, C. Tomas-Zapico, A. Costa, “Targeting beta-amyloid at the CSF: a new therapeutic strategy in Alzheimer's disease”, Hypothesis and Theory, Vol. 10 (100), Avril 2018; DOI: 10.3389/fnagi.2018.00100
M. Menéndez Gonzalez, “Mechanical Dilution of Beta-amyloid Peptide and Phosphorylated Tau Protein in Alzheimer's Disease: Too Simple to be True?”, Cureus. 2017 Feb. 28; 9(2):e1062. doi: 10.7759/cureus.1062.
Tõugu V., Tiiman A, Palumaa P, “Interactions of Zn(II) and Cu(II) ions with Alzheimer's amyloid-beta peptide. Metal ion binding, contribution to fibrillization and toxicity”, Metallomics. 2011 March; 3(3):250-61. DOI: 10.1039/c0mt00073f.
Cremades N., Dobson C. M., “The contribution of biophysical and structural studies of protein self-assembly to the design of therapeutic strategies for amyloid diseases”, Neurobiol Dis. 2018 January; 109(PtB):178-190.
doi: 10.1016/j.nbd.2017.07.009.
A. Czlonkowska, T. Litwin, P. Dusek, P. Ferenci, S. Lutsenko, V. Medici, J. K. Rybakowski, K. H. Weiss, M. L. Schilsky, Wilson disease, Nature Rev. Dis. Primers, 2018, 4:21. https://www.nature.com/articles/s41572-018-0018-3.
Pfaender S., Grabrucker A. M. Characterization of biometal profiles in neurological disorders. Metallomics. 2014 May; 6(5):960-77.
Boland B., Yu W. H., Corti O., Mollereau B., Henriques A., Bezard E., Pastores G. M., Rubinsztein D. C., Nixon R. A., Duchen M. R., Mallucci G. R., Kroemer G., Levine B., Eskelinen E. L., Mochel F., Spedding M., Louis C., Martin O. R., Millan M. J. Promoting the clearance of neurotoxic proteins in neurodegenerative disorders of ageing. Nat Rev Drug Discov. 2018 September; 17(9):660-688.
Iadanza M. G., Jackson M. P. Hewitt E. W., Ranson N. A., Radford S. E. A new era for understanding amyloid structures and disease. Nat Rev Mol Cell Biol. 2018 December; 19(12):755-773.

1. Device for the joint extraction of at least one metal cation and at least one target molecule from a biological fluid, a biological aggregate, an organ, or tissue, for diagnostic or therapeutic purposes, comprising:
at least one ligand exhibiting specific affinity for the target molecule; and
at least one means for extraction of the metal cation, said means being a perfusion fluid comprising at least one chelating agent, said perfusion fluid being contained in a dialysis or microdialysis system.

2. Device according to claim 1, wherein said means for extraction of the metal cation is a perfusion fluid used in a dialysis or microdialysis system, said perfusion fluid further comprising said ligand exhibiting specific affinity for the target molecule.

3. Device according to claim 1, wherein:
the complexation constant log(KC1) of the chelating agent for at least one metal cation is greater than 10, preferably greater than or equal to 15, and
said at least one cation is selected from the cations of metals Cu, Fe, Zn, Hg, Cd, Pb, Mn, Co, Gd, and Al, alone or in combination, and more particularly Cu, Fe, and Zn, alone or in combination.

4. Device according to claim 1 any, wherein said means makes it possible to extract the cations from a biological fluid, biological aggregate, organ, or tissue, when the content of said metal cations is less than 1 ppm.

5. Device according to claim 1, wherein said means makes it possible to extract a quantity of metal cations representing at least 1% of its mass.

6. Device according to claim 1 further comprising a dialysis system comprising a dialysis membrane and a reservoir comprising a perfusion fluid, said perfusion fluid being selected among:
a solution of nanoparticles comprising as active ingredient at least one chelating agent, and a solution of at least one ligand exhibiting specific affinity for the target molecule, the average diameter of said nanoparticles and of the ligand being greater than the pores of the dialysis membrane,
a solution of polymers, said polymers being grafted with at least one active ingredient which is a chelating agent, and a solution of at least one ligand exhibiting specific affinity for the target molecule, the average diameter being greater than the pores of said dialysis membrane.

7. Device according to claim 6, wherein the chelating agents are obtained by grafting, onto the nanoparticles or onto the polymer, one of the following complexing molecules: DOTA, DTPA, EDTA, EGTA, BAPTA, NOTA, DOTAGA, DFO, DOTAM, NOTAM, DOTP, NOTP, TETA, TETAM, TETP, and DTPABA, or mixtures thereof.

8. Device according to claim 6, wherein said nanoparticles are nanoparticles based on polysiloxane with an average diameter of between 3 and 50 nm, comprising the chelating agent obtained by grafting DOTA, DOTAGA, EDTA or DTPA onto the nanoparticles.

9. Device according to claim 1, wherein the chelating agent contains at least one alkaline earth cation, preferably a cation of metals selected among Ca and Mg.

10. Device according to claim 9, wherein at least 10% of the chelating agents of said device are precomplexed with an alkaline earth cation.

11. Device according to claim 6, wherein said nanoparticles are based on polysiloxane or said polymers are based on chitosan or polyethylene glycol or polyvinyl alcohols.

12. Device according to claim 1, wherein the target molecule is selected among proteins, peptides, glycoproteins.

13. Device according to claim 1, wherein the target molecule is selected among amyloidogenic proteins and components of amyloid structures.

14. Device according to claim 1, wherein the target molecule is selected among molecules involved in amyloidoses, tauopathies, or any pathology presenting a deposit based on one or more proteins.

15. Device according to claim 1, wherein the target molecule is selected among proteins and/or their precursors such as immunoglobulin light and heavy chains, serum amyloid A protein, transthyretin, apolipoprotein AI, AII, AVI, CII, or CIII, beta-2 microglobulin, gelsolin, lysozyme, fibrinogen, cystatin C, atrial natriuretic factor, calcitonin, amylin, insulin, prolactin, lactoferrin, cadherin, ABri, ADan, amyloid-beta peptide, prion protein, alpha-synuclein, tau protein, superoxide dismutase, huntingtin, neuroserpin, actin, ferritin, or mixtures thereof.

16. Device according to claim 1, wherein the ligand is an antibody or an engineered protein ligand of the target molecule.

17. Device according to claim 1, wherein the ligand is selected among:
antibodies targeting the amyloid-beta protein, preferably Solanezumab, Aducanumab, Crenezumab, Ponezumab, GSK933776, Gantenerumab, AAB-003, AAB-001, BAN2401, LY2599666, LY3002813, LY33887299322, SARI1014;
antibodies targeting alpha-synuclein, preferably BII054 or PRX002;
antibodies targeting the tau protein, preferably BII076, BII092, ABBV-8E12, JNJ-63733657, LY3303560, RG7345, R07105705, UCB0107;
antibodies targeting the serum amyloid A protein, preferably Dezamizumab or GSK2398852, Miridesap or GSK2315698, GSK3039294;
antibodies targeting transthyretin, preferably PRX004;
aptamers;
engineered protein ligands exhibiting specific affinity for at least one of the molecules, optionally selected among: ABD, Adhiron, Adnectin, Affibody, Affilin, Affimer, Affitin, Alphabody, Anticalin, Armadillo repeat proteins, Atrimer/tetranectin, Avimer/Maxibody, Centyrin, DARPin1;
engineered protein ligands of less than 50 kDA, preferably less than 30 kDa, and more preferably less than 5 kDa, grafted onto a nanoparticle or a polymer of more than 5 nm in hydrodynamic diameter, preferably more than 100 kDa, or
mixtures thereof.

18. A method of using a device according to claim 1 by providing the device to a patient presenting one of the following pathologies:
systemic and/or localized amyloidoses; preferably amyloidoses selected among: type AL amyloidosis (immunoglobulin light chain), type AH amyloidosis (immunoglobulin heavy chain), AA amyloidosis (serum amyloid A protein), ATTR amyloidosis (transthyretin), amyloid heart disease, renal amyloidosis, type II diabetes, prion diseases, or diseases linked to an amyloid protein;
tauopathies preferably selected among: Alzheimer's disease, progressive supranuclear palsy, frontotemporal dementia;
pathologies presenting a deposit based on at least one protein;
diseases presenting a metal dyshomeostasis preferably Wilson's disease or neurological disorders without amyloid characteristics such as autism or schizophrenia, . . . ), or neurological disorders with amyloid characteristics such as amyloidosis affecting or not affecting the central and/or peripheral nervous system, and
jointly extracting at least one metal cation and at least one target molecule from a biological fluid, a biological aggregate, an organ, or tissue of the patient.

19. Microdialysis system comprising a device for extraction according to claim 1 and such that it comprises at least:
a perfusion reservoir comprising perfusion fluid and a collection reservoir comprising the perfusion fluid comprising the extracted compounds or a combined reservoir comprising the perfusion fluid;
a two-way catheter connecting a microdialysis probe to the perfusion reservoir and collection reservoir or to the combined reservoir;
the microdialysis probe comprising a first lumen allowing the passage of perfusion fluid to a second lumen, said second lumen allowing discharge of the perfusion fluid comprising the extracted compounds, and a microdialysis membrane between the second lumen and the exterior of the microdialysis probe in contact with the biological fluid.

20. Dialysis system comprising a device for extraction according to claim 1 and such that it comprises at least:
a probe with separate lumens comprising a second catheter allowing biological fluid to enter the dialysis system and a first catheter allowing biological fluid to exit the dialysis system;
a reservoir comprising: i) perfusion fluid; ii) a dialysis compartment comprising a dialysis membrane separating the perfusion fluid from the biological fluid, the biological fluid entering said dialysis compartment via the second catheter and exiting via the first catheter.
